(19) United States
(12) Patent Application Publication
(10) Pub. No.: US 2023/0380759 A1
(43) Pub. Date: Nov. 30, 2023

(54) URINE ANALYZER TO MONITOR COMPLIANCE

(71) Applicant: Withings, ISSY LES MOULINEAUX (FR)

(72) Inventors: Sounderya NAGARAJAN, ISSY LES MOULINEAUX (FR); Christelle BARAKAT, ISSY LES MOULINEAUX (FR); David CAMPO, ISSY LES MOULINEAUX (FR)

(21) Appl. No.: 18/249,491

(22) PCT Filed: Oct. 20, 2021
(86) PCT No.: PCT/FR2021/051826

§ 371 Date: Apr. 18, 2023

(30) Foreign Application Priority Data
Oct. 21, 2020 (FR) 2010806

(51) Int. Cl. A61B 5/00; A61B 10/00

(52) U.S. Cl. CPC A61B 5/4833; A61B 10/007; A61B 5/742

(57) ABSTRACT

A urine analysis device for monitoring a user's compliance with a treatment including regular intake of a drug, the device including a collection channel for collecting the user's urine when the user urinates in a toilet and an analysis module configured to detect the presence of an analyte in the urine collected by the collection channel, the analyte being an active ingredient of the drug or a metabolite of the active ingredient, the analysis module being configured to determine a concentration of the analyte in the collected urine or a quantity related by a monotonic function to the concentration of the analyte in the urine.

### TECHNICAL AREA

The general field of the invention is that of devices for monitoring and improving therapeutic compliance with a drug treatment.

The invention relates more precisely to a urine analysis device adapted to monitor a user's compliance with a treatment comprising the regular intake of a drug. It also relates to a method of monitoring a user's compliance with his treatment using such a device.

### Previous Technique

In a 2018 study (OECD Health Working Paper No. 105, “Investing in medication adherence improves health outcomes and health system efficiency—Adherence to medicines for diabetes, hypertension, and hyperlipidaemia”), the OECD estimated that “failure to adhere to medication is estimated to be the cause of 200,000 premature deaths each year in Europe. People with major chronic diseases, in particular, are at serious risk of not taking their medication properly. Among diabetics and people with heart disease, patients who neglect their treatment in this way experience almost twice the mortality of patients who adhere rigorously to their treatment.”

Therapeutic adherence is therefore an important issue in order to provide better support to patients during their treatment, to improve the chances of success of their treatment, or in the context of clinical studies.

Devices such as connected medication boxes, whether or not associated with wearable devices (e.g., including an RFID tag or implementing NFC technology), are known to detect whether a patient is opening or has approached a medication box. Such devices, however, do not provide any certainty as to whether a patient has actually taken their medication.

When one wishes to detect the intake of a drug, one must go to a laboratory that will be able to perform an analysis on a biological fluid and detect the presence of the drug or a metabolite of it.

In addition to the costs, the need for qualified personnel and the sometimes invasive nature of these controls, laboratory measurements do not allow for a realistic estimate of compliance with a treatment for several reasons:
measurements are unique and it is difficult to estimate whether a drug has been ingested prior to the measurement;
it is easy to mislead the measurement if the patient only takes their treatment before going to the laboratory (false positive);
drugs with a short half-life may be cleared from the body prior to measurement (false negative);
drugs with a long half-life may result in a positive result when the treatment is no longer being followed (false positive); and
variability between patients and within the same day mean that the measurement must be taken at a specific time.

Thus, the presence of the drug or a metabolite of the drug only allows to deduce that the drug has been ingested in a given time interval before the measurement, which depends on the half-life of the drug and the patient. However, it is impossible to deduce from this measurement whether the patient is taking the drug regularly, especially over the long term.

It would be desirable to be able to monitor a user's adherence to his or her treatment over the long term in a simple, practical, reliable, inexpensive and non-invasive way.

### Presentation of the Invention

This is achieved by a urine analysis device for monitoring a user's compliance with a treatment comprising regular intake of a drug, the device having a collection channel for collecting the user's urine when the user urinates in a toilet and an analysis module configured to detect the presence of an analyte in the urine collected by the collection channel, the analyte being an active ingredient of the drug or a metabolite of the active ingredient, the analysis module being configured to determine a concentration of the analyte in the collected urine or a quantity proportional to the concentration (C) of the analyte in the urine.

By “quantity related by a monotonic function to the analyte concentration in the urine” is meant for example an optical quantity (e.g. fluorescence rate, absorbance rate, color, etc.) or a conductimetric quantity (e.g. conductance). The quantity can be, for example, proportional to the concentration at least over a concentration range. In the following and in the claims, when the analyte concentration is mentioned, this may also include any quantity measurable by the device that is related by a monotonic function to the analyte concentration.

The urine analysis device according to the invention makes it possible to monitor the intake of a drug in a simple and regular way, especially on a daily basis. It can indeed be configured to perform several successive analyses corresponding to several distinct micturitions of the user. The device is also non-invasive, since it can analyze urine without user intervention or complex sampling. The user only has to urinate in the toilet for the urine to be collected and analyzed by the device, and the results to be automatically transmitted to his doctor, an authorized organization or a relative. This device thus allows to improve the user's care during his treatment and the compliance for his treatment.

The urine collected through the collection channel comes directly from the user's own urination, i.e. the urine is not mixed with any other liquid before being collected, which makes the analysis more reliable and avoids any contamination of the urine with unwanted compounds.

In an embodiment, the drug may be a drug for treating a cardiac pathology selected from the following classes: diuretics (furosemide, amiloride, torsemide), statins (atorvastatin, pravastatin, lovastatin), anticoagulants (e.g. warfarin, enoxaparin, heparin, aspirin, clopidogrel, dabigatran, apixaban, rivaroxaban).

In an example embodiment, the drug may be a drug for treating hypertension selected from the following classes: calcium channel blockers (e.g., from the dihydropyridine family: lacidipine, efonidipine, clevidipine, cilnidipine, amlodipine, felodipine, nifedipine; or non-dihydropyridines: diltiazem, verapamil), angiotensin II antagonists (e.g., from the tetrazole family: candesartan, valsartan, losartan; or non-tetrazoles: eprosartan, telmisartan), converting enzyme inhibitors (e.g., captopril, ramipril, enalapril), diuretics (e.g., hydrochlorothiazide, indapamide, amiloride), and beta blockers (e.g., metoprolol, bisoprolol, atenolol, propranolol).

In one example embodiment, the drug may be a drug for treating epilepsy selected from the following classes: carbamazepine, phenytoin, or valproic acid.

In one embodiment, the drug may be an immunosuppressive drug (maintenance drugs) selected from the following classes: calcineurin inhibitors (e.g. tacrolimus, cyclosporine), antiproliferative agents (e.g. mycophenolic acid, mycophenolate mofetil, mycophenolate sodium, azathioprine), mTOR inhibitors (e.g. sirolimus), steroids (e.g. prednisone).

In one example embodiment, the analysis module may be configured to, at each analysis, contact the collected urine with a detection compound capable of binding to the analyte and to detect the presence of the complex between the analyte and the detection compound.

The detection compound is capable of selectively binding to the analyte. The detection compound can be detected directly (e.g., by a change in color, fluorescence, or conductance of the solution when the detection compound binds to the analyte), or indirectly by using a label (e.g., a fluorescent or electrochemically detectable label) that can bind to the complex formed by the detection compound and the analyte.

In one example embodiment, the sensing compound is a Molecularly Imprinted Polymer (MIP).

When the detection compound is a MIP, it may change its optical or electrochemical property when it binds to the analyte. For example, the MIP may become fluorescent when it binds to the analyte. For example, the conductance of a solution comprising the MIP may change when the MIP binds to the analyte. Alternatively, a fluorescent or electrochemically detectable label may be used that detects the complex formed by the MIP and the analyte.

A detection compound in the form of a MIP is advantageous in that it is more stable at room temperature than an aptamer, antibody or protein (other known detection compounds). This is particularly useful when this compound needs to be stored in a device positioned in the toilet for a significant period of time, for example when monitoring a long-term treatment. Furthermore, a MIP can be stored in solution or in solid form. Also, the MIP can be used to perform a quantitative measurement of analyte concentration in urine because its properties change when it binds with the analyte. Finally, MIP is less expensive and easier to obtain than an aptamer or an antibody.

Furthermore, it is possible to design a MIP that is capable of forming a complex with several different analytes, for example with the active ingredients of drugs of a particular drug class (i.e., a set of molecules having similar or identical portions). Thus, the use of a MIP makes the device according to the invention even more versatile.

In an example embodiment, the analysis module can be configured to further measure a correction quantity of the analyte concentration that is characteristic of the user's hydration, for example the correction quantity is selected from: creatinine concentration in the collected urine, specific gravity of the collected urine, urinary flow rate of micturition, conductivity of the collected urine.

The analyte concentration measured during an analysis can thus be corrected by means of the correction variable. The hydration of a user can vary during the day and from day to day, which influences the volume of urine during micturition and the concentration of analyte in the urine independently of the amount of medication taken. This arrangement allows to improve the result of the analysis and to be able to compare several successive analyses taking into account the hydration of the user.

In one example embodiment, the analysis module may comprise a plurality of test strips and/or a microfluidic chip.

An analysis module comprising test strips is advantageous in that it is simple and inexpensive to use. Furthermore, it is possible to test several analytes with one strip to detect the intake of several different drugs, or to use several strips each of which can detect the intake of different drugs. Test strips can be used to measure the correction variable of analyte concentration.

In one example embodiment, the test strip may be a colorimetric test strip or a lateral or vertical flow immunoassay test strip. The fluorescence level of a portion of the test strip may, for example, be directly related to the analyte concentration in the urine.

In an example of an embodiment, the analysis module may comprise a plurality of test strips and a rotatable holder on which the strips are fixed, said holder being removable from the urine analysis device. Such a device allows for a plurality of test strips on a single holder, which can be changed as a consumable and allows for regular monitoring of medication intake.

In an example embodiment, where the analysis module comprises a microfluidic chip, urine and detection compound solution can be delivered as drops, the microfluidic chip is then adapted to merge drops of urine and detection compound solution at the mixing zone. In this case, the presence of the complex between the analyte and the detection compound can be detected in the merged drops (discretely).

Alternatively, the urine and the detection compound solution can be delivered as continuous streams that are mixed at the mixing zone to obtain a single stream. In this case the presence of the complex between the analyte and the detection compound can be detected in the single stream (continuously).

A microfluidic chip uses only small amounts of sensing compound solution, saving it and allowing a large number of analyses to be performed. In particular, the use of a microfluidic chip with a detection compound in MIP form is particularly advantageous for these reasons. In addition, the same microfluidic chip can be used to detect the presence of several different analytes. It should be noted that the detection compound can be stored in solid form and then dissolved before being injected into the chip, or stored directly in solution.

In one example embodiment, the microfluidic chip may be removable from (and thus be a consumable of) a device case or fixed in the device case. The case may include means for recharging the device with sensing compound.

In one embodiment, the urine analysis device may indeed comprise a case configured to be removably positioned in the toilet, the analysis module being contained within said case. With such an arrangement, the urine analysis device can be transported, for example, provided or delivered directly to a user upon request of his physician or an authorized organization when he starts his treatment, and then be returned if needed. Such a device allows to start the treatment follow-up immediately. The installation of the device in a toilet is also facilitated. For example, the device can be installed directly in a user's toilet without having to modify the design of the toilet. The removable nature of the case makes it possible, for example, to change a consumable in the analysis module, to modify the analysis module to be able to detect another analyte, to install it in other toilets, or to recharge the device when it is powered by a battery. This makes the device versatile and modular.

In an example embodiment, the case may be configured to be installed entirely within a toilet bowl. In an example embodiment, the device may further comprise a pumping system for conveying urine from the collection channel to the analysis module.

In one embodiment, the case may have a front face for directly receiving a stream of urine from the user sitting on the toilet, a rear face opposite the front face, and a collection port on the front face or rear face that is in fluid communication with the collection channel. The user's urine can run down the front face and possibly the rear face to reach the collection port. Such a device is very simple and discreet to use, and allows to collect enough urine without air bubbles to perform the analysis. Furthermore, the user does not have to think about the urine analysis device to have his urine collected, allowing a better monitoring of the medication intake at home in a non-invasive way.

In one example embodiment, the analysis module may comprise an optical sensor and/or an electrochemical sensor. In particular, the type of sensor depends on the detection compound that is used.

In an example embodiment, the device may further comprise a communication module configured to transmit the analyte concentration or a test result to a remote device, such as a cell phone and/or a remote server. In this way, the analysis result can be transmitted automatically, and a physician or relevant organization can be informed in real time.

The invention also relates to a system for monitoring the therapeutic compliance of a user following a treatment comprising the regular intake of a medication, the system comprising a urine analysis device such as the one described above, and a processing unit for comparing the analyte concentration in the collected urine with at least one predetermined threshold in order to obtain a result as to the follow-up of the treatment. The device may comprise the processing unit, or the processing unit may be remote. The at least one predetermined threshold may be obtained from a custom calibration step or a learning step.

The result may be, for example, that the user has missed one or more doses of his medication or that he is overdosed with respect to his prescription.

The result can also be a measurement error if the measured analyte concentration is below a low error threshold or above a high error threshold.

The invention also relates to a method for monitoring the therapeutic compliance of a user following a treatment comprising the regular intake of a medication, using a urine analysis device such as the one described above, the method comprising:
a) performing at least one analysis corresponding to at least one micturition of the user by following the following steps:
a1) collecting urine from a user urinating in the toilet through the collection channel of the urine analysis device, and
a2) analyzing the collected urine by the analysis module to obtain an analyte concentration in the collected urine; and
b) obtaining a treatment monitoring result from the obtained analyte concentration.

In an example embodiment, the method may further comprise a step a3) of obtaining an analyte concentration corrected for user hydration, e.g. the analyte concentration is corrected by a creatinine concentration in the collected urine, by a specific gravity of the collected urine, by a urinary flow rate of the micturition, by the conductivity of the collected urine.

In one example embodiment, step b) may comprise: comparing the analyte concentration (or corrected analyte concentration) in the collected urine to at least one predetermined threshold. By “compare” is meant checking whether the analyte concentration is below or above the predetermined threshold. In addition, it can be checked whether the analyte concentration is below a first (low) threshold and/or above a second (high) threshold. For example, if the concentration is below the first threshold, it can be inferred that the user has missed a dose, or if the concentration is above the second threshold, it can be inferred that the user is overdosing.

In one example embodiment, the method may include a custom calibration pre-step during which a plurality of analyte concentrations corresponding to different micturitions of the user while undergoing treatment according to a prescription are obtained, and the predetermined threshold is calculated from the obtained concentrations. The predetermined threshold may be obtained from the sample mean and standard deviation of the concentrations obtained in the calibration step.

Preferably, the calibration step can be performed using a device according to the invention.

This preliminary step of personalized calibration or learning allows to personalize the compliance monitoring to the user in order to increase its reliability.

This calibration step makes it possible to take into account variations in the concentration of analyte in the urine during the same day, but also over several days.

It can also be used to better understand the user's response to the drug, especially to the prescribed dosage.

In one example embodiment, the predetermined threshold may be a lower control limit (LCL) or an upper control limit (UCL) calculated on the sample of concentrations obtained in the calibration step. For example, if the analyte concentration is below the lower control limit, it can be inferred that the user has missed a dose of their medication. For example, if the analyte concentration is above the upper control limit, it can be inferred that the user has taken too much drug (“overdose”), or that there has been an interaction with another drug.

In an example embodiment, the method may include sending or displaying a notification when the result is that the user did not follow their treatment correctly (i.e., they were not compliant with their treatment).

In one example embodiment, the content of the notification may depend on a rate of change of analyte concentration between at least two successive analyses and the half-life of the drug.

### BRIEF DESCRIPTION OF THE DRAWINGS

Further features and advantages of the present invention will be apparent from the description below, with reference to the attached drawings which illustrate non-limiting examples of its implementation. In the figures:

FIG. 1 is a diagram showing schematically the different parts of a device according to an embodiment of the invention.

FIG. 2 is a schematic view of a device according to a first embodiment of the invention positioned inside a toilet bowl.

FIG. 3A and FIG. 3B are front and rear views, respectively, of the device according to the first embodiment.

FIG. 4A and FIG. 4B are perspective views of the interior of the device according to the first embodiment from the front.

FIG. 5 shows a perspective view of a rotatable holder comprising test strips present inside the device according to the first embodiment.

FIG. 6 schematically shows an example of a microfluidic chip that can be used in a device according to a second embodiment of the invention.

FIG. 7 shows the different steps of a process for monitoring a user's compliance with medication.

FIG. 8 is a graph showing concentrations obtained during the calibration step.

FIG. 9 shows the different steps to obtain an analysis result.

FIG. 10 shows three graphs giving the concentrations measured during a treatment follow-up as a function of time corresponding respectively to a normal situation, a missed intake situation and an overdose situation.

FIG. 11 schematically illustrates the use of a device according to an embodiment of the invention with a cell phone and/or a server.

FIG. 12 shows an example of a user's smartphone app that allows them to track their treatment and an example of a physician's app that allows them to track their patient.

### DETAILED DESCRIPTION OF THE METHODS OF IMPLEMENTATION

General description of the urine analysis device.

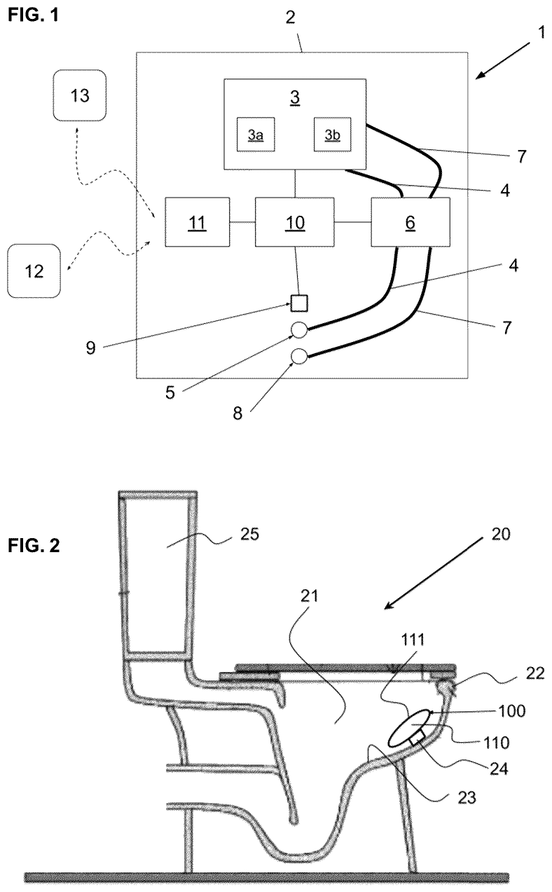

FIG. 1 schematically illustrates the various parts of a urine analysis device 1 according to one embodiment of the invention.

The device 1 may generally comprise: a case 2 in which an analysis module 3 is present, a collection channel 4 for conveying urine from a collection port 5 to the analysis module 3, a pumping system 6 for conveying urine into the collection channel 4, a purge channel 7 for discharging urine after an analysis through a purge port 8, a urine detector 9 (e.g. a thermistor) to automatically start an analysis when urine is detected in the vicinity of the collection port 5, a control unit 10 to control the various elements of the device 1, and a communication module 11 to communicate with a device 12 such as a smartphone or a remote server. The device 1 can be powered by a battery (not shown).

The collection port 5 may be located on one face of the case 2 to collect urine, or it may be connected to a trough or arm extending from one face of the case 2 to collect urine directly when the user urinates in the toilet.

Advantageously, the case 2 is configured to be removably positioned in a toilet, for example by means of a suction cup attached to a toilet wall or a hook attached to the rim of the toilet. The case 2 may be placed entirely within the toilet bowl, making it unobtrusive and increasing its acceptability to a user. According to an example embodiment, the case is contained in a parallelepiped with dimensions 110 mm×110 m×50 mm. According to an embodiment, the case has a general discoidal shape with a diameter between 80 and 100 millimeters and a thickness between 40 and 50 millimeters.

The communication module 11 is wireless, and may use, for example, one or more of these technologies: Bluetooth, Low-Energy Bluetooth (BLE), Wi-Fi, GSM, 3G, 4G/LTE(M), and/or 5G. The communication module 11 can connect to the Internet directly, via a local network with a wireless router or hub connected to the cell phone network.

The device 1 may be provided with a remote button 13, communicating for example wirelessly with the communication module 11. The remote button 13 may be provided with means for identifying the user, such as a biometric sensor (e.g., a fingerprint reader), and with display means (e.g., a screen and/or colored LEDs) to indicate that a user has been recognized. The analysis can then be triggered automatically without user intervention if urine is detected by the urine sensor 9 and a user has been identified. Alternatively, the user's smartphone or a connected bracelet (such as a connected watch) can be used to identify the user and trigger an analysis. In the case of toilets used by multiple people, the means of identification of a particular user can disregard a measurement performed for another person. The monitoring of the medication intake is therefore totally personalized.

The analysis module 3 may comprise a storage area 3a in which a detection compound is present for detecting the analyte when it binds thereto, and a detection module 3b for detecting the presence of a complex formed between the detection compound and the analyte (and optionally a label).

The analysis module 3 is adapted to contact urine collected through the collection channel 4 with the detection compound to allow detection of the complex formed between the detection compound and the analyte by the detection module 3b.

The detection module 3b may include, for example, an optical sensor (such as a photodiode or CCD sensor) and/or an electrochemical sensor (such as a biosensor, potentiostat, galvanostat, amperometric sensor, or polarographic sensor).

The analysis module 3 can further be configured to measure an analyte concentration correction variable that is characteristic of the user's hydration, such as creatinine concentration in the collected urine, urine specific gravity, urine conductivity, or urinary voiding rate. The correction of the analyte concentration will be described in more detail later.

The analyte to be tested is characteristic of the user's intake of a drug, i.e., it is present in the urine when the user has taken the drug and absent when the is user has not. For example, the analyte may be an active ingredient in the drug or a metabolite of the active ingredient.

The detection compound can advantageously be a Molecularly Imprinted Polymer (MIP) capable of specifically binding to (or capturing) the analyte to be detected.

A MIP for detecting a particular analyte can be made from crosslinkable functionalized monomers that are copolymerized in the presence of the analyte, the analyte then forms a fingerprint molecule. A MIP can thus be made from one or more given analytes.

The MIP can be functionalized with a fluorescent label that activates when the MIP is bound to the analyte (or in other words, whose fluorescence increases or decreases when the MIP is bound to the analyte), or with an electrochemically detectable label that activates when the MIP is bound to the analyte (e.g., the conductance of a solution containing the MIP can vary when it binds to the analyte). It is then possible to directly detect the presence of a second compound constituted by the MIP/analyte complex. Other uses of a MIP are of course possible to detect the analyte.

A fluorescent marker can be selected for example from: nanoparticles (e.g. quantum dot), a quencher probe such as QSY®9, 4-Amino-1,8-naphthalimide, a dye of the boron-dipyrromethene family (“BODIPY”).

A marker detectable by electrochemical measurement can be selected for example from: graphene oxide nanoparticles, gold nanoparticles, boron-doped diamond.

The analysis module 3 may include test strips 121, as in the device shown in FIGS. 2 through 5, or a microfluidic chip 30 such as that shown in FIG. 6. The choice between these two solutions may depend on the analyte to be tested, the detection compound that is used, and/or the detection method (optical and/or electrochemical for example).

A therapeutic compliance monitoring system may include a device 1 and a processing unit that compares an analyte concentration obtained by the analysis module to a predetermined threshold in order to deduce whether the user has followed his treatment according to a prescription. The processing unit can be present in the device 1 or remote, for example in a remote server or a cell phone.

### First Embodiment—Test Strips

In FIG. 2, a urine analysis device 100 according to a first embodiment of the invention positioned in a toilet 20 is shown. The toilet 20 generally comprises a bowl 21, bounded by a rim 22, and having an inner wall 23. The device 100 comprises here a single case 110 removably attached to the inner wall 23 of the toilet bowl 21 by means of a suction cup 24 and magnets attached to the suction cup and within the case 110. The case 110 is attached to the portion of the inner wall 23 facing the toilet flush tank 25 of the toilet 20. The case 110 is advantageously present entirely in the toilet bowl 21, i.e., it does not protrude from the rim 22 of the bowl 21, which makes it discreet and thus improves the monitoring of the user who is not disturbed by the presence of the device 100.

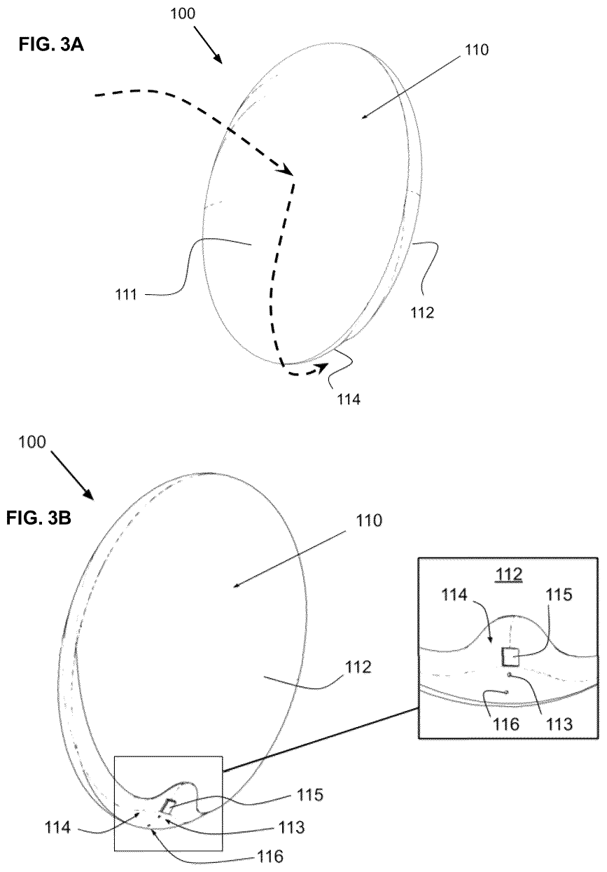

As can be seen in FIG. 3A and FIG. 3B, the case 110 of the device 100 here has a front face 111 and a rear face 112, and generally takes the form of a circular pebble. In this example, a collection port 113 is present in a recess 114 (negative relief) located on a lower end (relative to a normal position of use) of the rear face 112 of the case. Thus, the user can urinate on the front face 111 of the device while sitting on the toilet, and the urine can trickle down and be channeled to the collection port 113. An example of a urine path when a user urinates on the device 100 is schematized by dotted arrows in FIG. 3A. The urine can then be detected by the urine sensor 115 located near the collection port 113 and an analysis can be initiated (e.g., if a user has been previously identified). A purge port 116 is also present in the recess 114 below the collection port 113 to drain off excess urine after an analysis.

In an alternative embodiment not shown, the collection port 113 may be present on the front face 111 and a positive or negative relief allows urine received on the front face 111 to be channeled to the collection port. The positive or negative relief allows for better collection by channeling urine, but is not essential to achieve collection in the described embodiments.

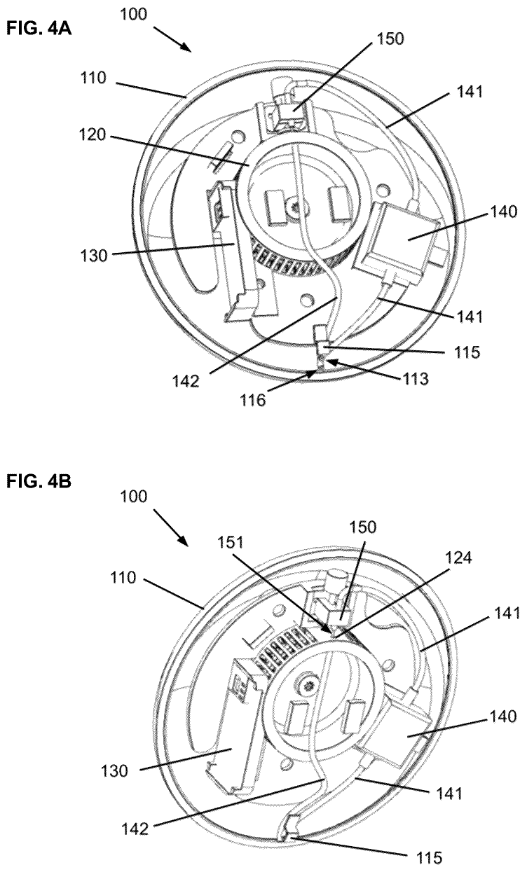

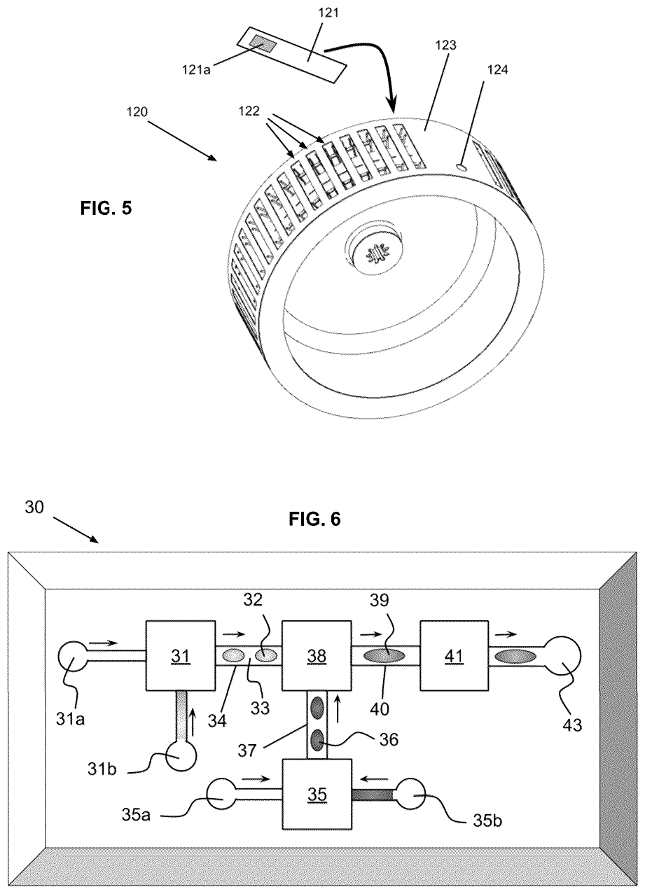

FIG. 4A and FIG. 4B show various components within the case 110 of the device 100. In this embodiment, the analysis module comprises a removable rotatable holder 120 to which test strips 121 are attached (FIG. 5), and a detection module comprising an optical sensor 130 capable of detecting a color change (e.g., by absorbance or fluorescence, by illuminating the strip 121 with an appropriate light) on a test strip 121. A pump 140, for example a piezoelectric pump, is used to deliver urine from the collection port 113 to an injector 150 via a collection channel 141. The injector 150 is provided with an automated syringe 151. The injector 150 is used to inject a controlled amount of urine onto a test strip 121 when the device 100 is in an injection position, or to discharge urine through the purge channel 142 when in a purge position (as in FIG. 4A and FIG. 4B). The various channels of the device may be hydrophobic to avoid contamination between different analyses.

As can be seen in FIG. 5, the rotatable holder 120 here takes the form of a hollow cylinder or ring to which the test strips 121 are attached. In this example, the test strips 121 are fixed in housings 122 present on the outer surface 123 of the rotatable holder 120. The strips 121 are isolated from each other and from the external environment to allow for storage. The rotatable holder 120 comprises here an opening 124 that can be passed through by the syringe 151 of the injector 150 in the purge position. The rotatable holder 120 coupled to a stepper motor located in the case 110 (not visible in the figures) allows a test strip 121 to be selectively moved from the injection position where urine can be deposited therein, to an analysis position where the optical sensor 130 can detect a change in color of the strip 121 to detect the presence of an analyte. The rotatable holder 120 may be removable from the case 110, i.e., may be a consumable that can be replaced, for example, when all of the test strips 121 have been used.

In an example not shown, the strips 121 may be present on an inner side of the rotatable holder 120, and if an optical measurement is used, it may be performed by transmission, for example.

The test strips 121 generally include a storage area 121a for the detection compound, in which the compound may be stored in solid form, for example. The test strips 121 may be conventional colorimetric strips or lateral or vertical flow immunoassay strips. A MIP may be used in a test strip 121.

A conventional colorimetric strip may include a pad containing the detection compound which, when soaked in urine, may change color if the analyte is present in sufficient concentration.

In a lateral flow or vertical flow immunoassay strip, urine deposited on the strip can migrate by capillary action to a test area or line where the detection compound is bound and can bind to the analyte to form a detectable complex. Eventually, depending on the configuration used, a marker may also be present on the strip to allow detection.

The test strips 121 can be used to measure a correction variable for analyte concentration in urine, such as creatinine concentration or urine specific gravity.

Second embodiment—microfluidic chip.

FIG. 6 shows schematically an example of a microfluidic chip 30 that can be used in an analysis module 3 of a urine analysis device 1 according to a second embodiment of the invention. The microfluidic chip 30 can be used in a case 110 such as the one described above to benefit from a simple and air bubble-free urine collection by trickling the urine over the case to the collection port.

The principles of microfluidics with drops are used in this example. In FIG. 6, the arrows symbolize the direction of fluid flow in the chip 30 and the circles represent the points of entry or exit of the fluids into the chip. The chip 30 can be made of, for example, ceramic such as glass, polymer such as polydimethylsiloxane (PDMS), or any other suitable material.

In this example, the chip 30 comprises a generator 31 of first drops 32 of urine that is supplied on the one hand with carrier fluid (e.g., mineral oil) through a first injection point 31a and with urine through a second injection point 31b. The second injection point 31b may be in direct or indirect fluid communication with the urine collection channel of the urine analysis device. The generator 31 generates the first drops 32 of urine in a first channel 34.

The chip 30 further comprises a generator 35 of second drops 36 of detection compound in solution, which is supplied on the one hand with carrier fluid 33 via a first injection point 35a and on the other hand with detection compound solution via a second injection point 35b in fluid communication with a reservoir of detection compound in solution (and optionally of a marker). The generator 35 generates the second drops 36 of detection compound in solution in a second channel 37. It should be noted that the detection compound may be stored in solution, or in solid form and then dissolved before being injected into the chip 30.

The droplet generators 31 and 35 can be of any type known to the skilled person, and for example implement the “flow focusing” technique.

The first channel 34 and the second channel 37 then meet in a mixing zone 38 in which the first drops 32 can merge with the second drops 36 to form third drops 39. The third drops 39 exit the mixing zone 38 into a third channel 40. The mixing zone 38 may include any type of means for fusing two drops in a carrier fluid that are known to the skilled person. In particular, electrodes or particular channel geometries may be used to allow two consecutive drops to merge.

After traveling through the third channel 40, the third drops 39 may pass through a detection zone 41 where a sensor (e.g., optical or electrochemical—not shown) may detect the presence of the second compound formed by contacting the detection compound and the analyte in a third drop 39.

Finally, all fluids leaving the detection area 41 can be discharged from the chip 30 through an outlet 43 connected to the purge channel 7 of the urine analysis device 1.

It is possible to use a microfluidic chip in which not drops, but continuous streams of urine and sensing compound solution are mixed into a single stream that is continuously analyzed by one or more sensors.

Of course, other microfluidic chip configurations are possible. In particular, multiple analytes could be tested by adding additional mixing and detection areas on the same chip. Multiple microfluidic chips can be used. Both an optical and an electrochemical sensor can be used with a single microfluidic chip.

The microfluidic chip 30 can further be configured to measure urine creatinine concentration or urine specific gravity using corresponding reagents.

Method for Monitoring a User's Compliance with a Medication.

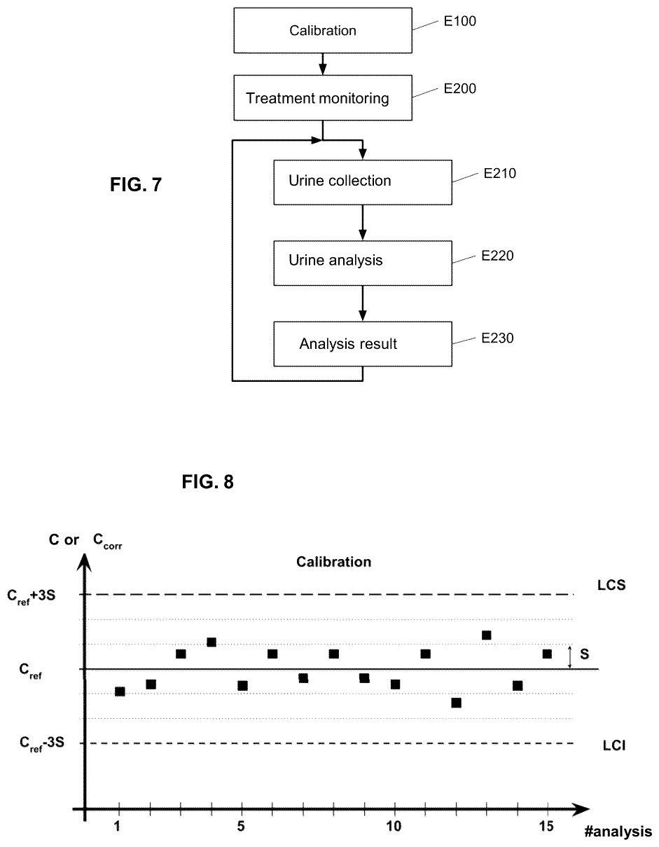

FIGS. 7 through 10 illustrate a method of monitoring a user's compliance with a medication according to one embodiment of the invention.

A prior personalized calibration step E100 is performed during which the urine collected from several distinct micturitions of the user is successively analyzed when the user follows his treatment correctly, i.e. according to his prescription. The analyses can be performed at random times of the day, during a predetermined period of time. For this purpose, the device 1 can be used to obtain the analyses of the calibration step, and can be positioned in the user's toilet who only has to urinate on it. The duration of the calibration step can be determined by a prior clinical trial.

The calibration step can be started after a predetermined time depending on the half-life of the drug, in order to perform the calibration when the analyte concentration in the urine is steady-state, i.e. oscillating around a mean concentration. For example, the calibration phase can be started after a time since the beginning of the treatment that is more than four or five times the half-life of the drug in the urine.

During this step E100, a plurality of analyte concentrations C are obtained.

These concentrations C can be corrected to C_{corr} to account for the user's hydration and improve the reliability of the process, for example:
by using the creatinine concentration C_{creat} in the urine: C_{corr}=C/C_{creat}
by using the specific gravity S_{g} of the urine: C_{corr}=C.(S_{g}−1)/(S_{g}+1),
using the conductivity of the urine, or
using the urine flow rate U: C_{corr}=a-b.log(U); a and b being analyte dependent constants to be determined experimentally.

FIG. 8 shows an example of analyte concentrations measured by the device 1 during a calibration step (here 15 measurements were obtained). In this example, a reference concentration C_{ref} is calculated, corresponding to the average of the concentrations C or C_{corr} obtained during the calibration step, and a standard deviation S, making it possible to calculate, for example, an upper control limit LCS=C_{ref}+3S and a lower control limit LCI=C_{ref}−3S. Of course, other statistical variables can be used depending on the drug considered and the sensitivity chosen. For example, alternatively LCS=C_{ref}+2S and LCI=C_{ref}−2S can be used, or LCS=C_{ref}+S and LCI=C_{ref}−S.

Then, once the calibration has been carried out, the treatment is monitored (step E200). To do this, the device 1 is positioned in the user's toilet, and the user only has to urinate on it. The urine is collected (step E210) automatically during urination, and analyzed (step E220) automatically by the device 1, and possibly a remote processing unit to which the device 1 will have sent the result of its concentration measurements.

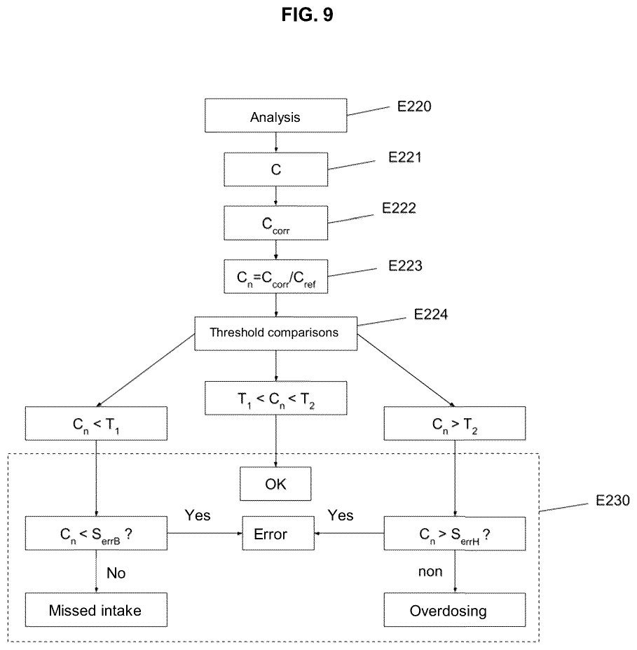

FIG. 9 details an example of processing performed during an analysis (E220). An analyte concentration C is obtained at each analysis (E221), and can be corrected to obtain a concentration C_{corr} (E222) as described previously. The use of the corrected concentration C_{corr} allows, among other things, to reduce false alarms that would be due to dehydration or too much hydration of the user (overdose or missed dose alarms). The corrected concentration C_{corr} can then be normalized by the concentration C_{ref} (E223) obtained during the calibration phase, in order to allow easier data processing.

Then, the normalized corrected concentration Cn=C_{corr}/C_{ref} obtained is compared (E224) with a first low threshold (T=LCI/C_{1ref} in this example) and/or a second high threshold (T=LCS/C_{2ref} in this example). Other thresholds can be used depending on the chosen sensitivity and the drug.

The result of the analysis (E230) is obtained based on the comparison of the concentration Cₙ with the selected predetermined threshold(s):
If Cₙ is between T₁ and T₂, the analyte concentration in the urine is considered to correspond to a correct treatment follow-up.
If Cₙ is less than T₁, it is checked that it is not less than a predetermined low error threshold S_{errB}, which would signal an error in the measurement. For example, S_{errB} may correspond to the theoretical concentration that would be obtained after two successive missed shots. If this is not the case, it can be inferred that there was a missed drug intake. A decay rate t1=ΔCₙ/Δt between the concentration obtained at this test and the previous test can be calculated to assess, based on the half-life of the drug in the urine, whether the user should take the drug immediately or wait for the next intake. For example, a notification can be sent to the user or to his physician.
If Cₙ is greater than T₂, it is checked that it is not greater than a high error threshold S_{errH}, which would indicate an error in the measurement or a drug interaction requiring further investigation. For example, S_{errH} may correspond to the theoretical concentration that would be obtained after two simultaneous doses of the drug. If this is not the case, it can be deduced that there has been an overdose resulting from at least two drug intakes too close to the prescription. A growth rate t2=ΔCₙ/Δt between the concentration obtained at this test and the previous test can then be calculated to assess, based on the half-life of the drug in the urine, the time of the additional intake and the associated risk for the user. For example, a notification can be sent to the user or to the user's physician.

The error thresholds S_{errB} and S_{errH} can be determined during the calibration phase.

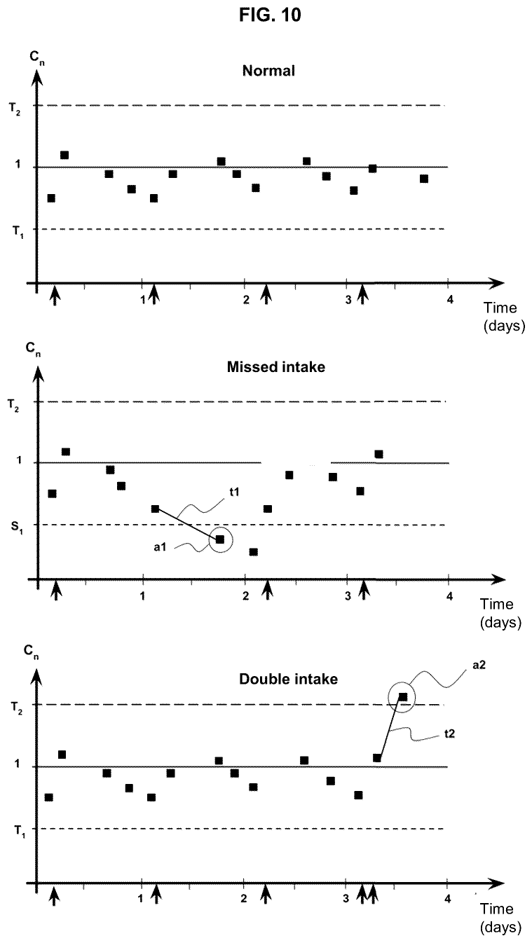

FIG. 10 shows three examples of the evolution of the concentration Cₙ as a function of time, corresponding to three behaviors of a user with respect to his treatment: a normal follow-up of his treatment (observant user), a missed drug intake (non-observant user), and a double drug intake (overdosed user). Each graph represents Cₙ as a function of time over four days. Arrows pointing to the x-axis indicate the time of drug intake.

In the “normal” case, it can be seen that the concentrations Cₙ obtained are always between the low T₁ and high T₂ thresholds, meaning that the user is following his treatment according to the prescribed scheme.

In the case of a “missed intake” on day 2, we can see that Cₙ is lower than the low threshold T₁ at the end of the day in the analysis referenced a1, triggering a missed intake alert. The rate of decrease of Cₙ compared to the previous analysis (shown by the segment t1) allows to deduce that the intake was missed the same morning, and a notification can be sent to the user.

To increase the reliability of missed dose detection, one can wait until two successive Cₙ values are below the low threshold T₁. This avoids sending a notification when the user has taken the medication with a delay compared to the usual time of taking.

In the case of a “double intake” on day 3, we can see that Cₙ exceeds the high threshold T₂ in the second part of the day, triggering an overdose alert. The growth rate of Cₙ compared to the previous analysis (shown as segment t2) allows to deduce that the two intakes took place the same morning, and to trigger an alert accordingly. The user can be invited (for example with a notification) to get closer to his doctor, and not to take any medication before a next normal analysis or before a predetermined duration.

Other thresholds to detect a missed dose or an overdose can be used and determined from a personalized calibration step. It is also possible to define intermediate thresholds allowing, for example, to detect a drug intake that is out of time with respect to the time foreseen by the prescription (both early and late).

Alternatively, the custom calibration phase may include determining thresholds for inferring missed dose or overdose from a pharmacokinetic curve of the drug in the urine and user-related data, i.e., without using the urine analysis device.

Alternatively, in the custom calibration phase, the thresholds can be determined both from a plurality of analyses of the user's urine while taking the treatment as prescribed, from a pharmacokinetic curve of the drug in the urine, and from user-related data.

The analysis result, if obtained directly by the urine analysis device 1, may be transmitted via the device's communication module 11 to a remote device 12, such as a smartphone 13 and/or a remote server 14 (FIG. 11). The transmission of the analysis result may allow the smartphone 13 and/or server 14 to generate a notification 15 to the user, a physician 16 or an authorized agency 17. If the analysis result is determined by a remote processing unit, for example in the smartphone 13 or remote server 14, the latter may generate and send the notification.

Example of a mobile application for monitoring medication compliance.

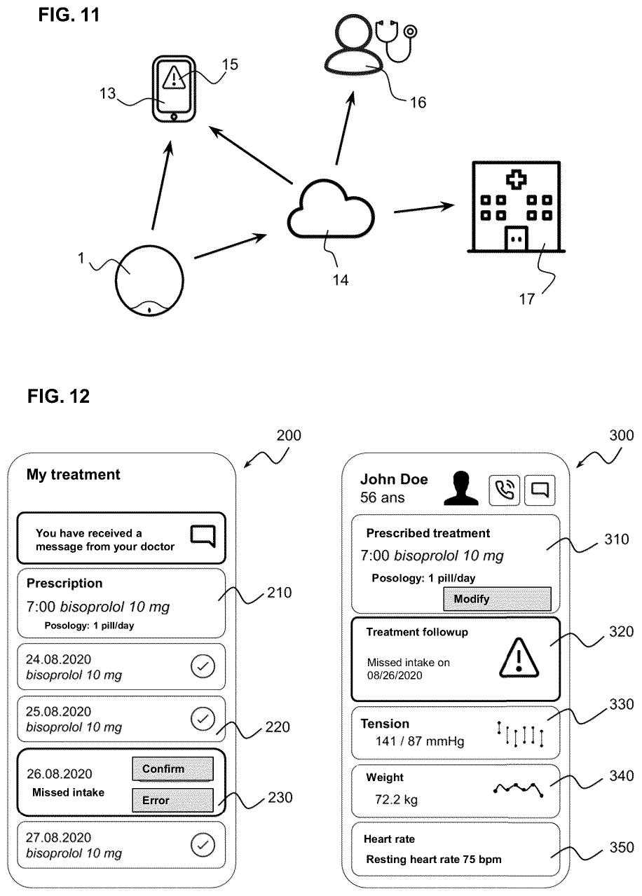

FIG. 12 shows, at left, an example of an application 200 for a user's smartphone. The application 200 may allow the user to view his or her prescription (210), the various intakes of the prescribed medication (220) as determined by an analysis device according to the invention, and report when the user has not taken his or her treatment (230). The application can integrate a set of automated recommendations (“coach”) to help the user better follow his treatment.

FIG. 12 shows, on the right, an example of an application 300 that could be used by a physician or an authorized organization to track a patient's treatment.

The application 300 may aggregate useful information to track the user's health status, such as: prescribed treatment (310); treatment tracking (320) with a message when a dose has been missed; blood pressure (330) as measured by the physician or at home by a connected blood pressure monitor; weight (340) as measured by the physician or at home by a connected body scale; or resting heart rate (350) as measured, for example, by a connected watch.

More generally, the device can be connected to a telemedicine platform allowing remote monitoring of a patient by a doctor or an authorized organization.

The user can thus have better visibility on his treatment, and be better monitored by his doctor or an authorized organization. Thus, with the urine analysis device according to the invention and such tools, the patient's compliance with his treatment can be improved.

Other Applications.

A device 1 according to the invention can further be used to more accurately characterize a user's response to a drug (including its elimination), or to study possible interactions between his treatment and other drugs. The treatment of the user, in particular the drug, the amount of active ingredient and the dosage, can be customized, for example, based on the data collected by the device.

Unusual variations in urine analyte concentration compared to a history may also detect kidney disease.

1-26. (canceled)

27. A system for monitoring compliance of a user with a treatment comprising regular intake of a drug, the system comprising a urine analysis device and a processing unit, wherein the system is configured to:
(a) perform at least one analysis for at least one micturition of the user by:
a1) collecting urine from a user urinating in a toilet through a collection channel of the urine analysis device, and
a2) analyzing the urine collected by an analysis module of the urine analysis device to obtain an analyte concentration in the collected urine or a quantity related by a monotonic function to the analyte concentration in the urine, wherein the analyte is an active ingredient of the drug or a metabolite of the active ingredient; and
(b) obtain a result by the processing unit as to a follow-up of the treatment from the analyte concentration or the quantity obtained.

28. The system of claim 27, wherein the processing unit is part of the urine analysis device.

29. The system of claim 27, further configured to: a3) obtain a corrected analyte concentration to account for user hydration

30. The system of claim 29, wherein the analyte concentration is corrected by a creatinine concentration in the collected urine, by a specific gravity of the collected urine, by a urinary flow rate of the micturition, or by the conductivity of the collected urine.

31. The system of claim 27, wherein, to obtain the result, the system is configured to compare the analyte concentration in the collected urine to at least one predetermined threshold.

32. The system of claim 31, further configured to perform a prior personalized calibration during which a plurality of analyte concentrations are obtained corresponding to different micturitions of the user when the user follows the treatment in accordance with a prescription, and the predetermined threshold is calculated from the concentrations obtained.

33. The system of claim 32, wherein the predetermined threshold is a lower control limit or an upper control limit calculated on the sample of concentrations obtained in the calibration step.

34. The system of claim 27, further configured to send or display a notification when the result is that the user has not completed the treatment correctly and a content of the notification depends on a rate of change of the analyte concentration between at least two successive analyses.

35. The system of claim 27, wherein the urine analysis device comprises a case configured to be removably positioned in the toilet, the analysis module being contained within said case, the case comprising a collection port for collecting urine dripping onto the case.

36. The system according to of claim 27, wherein the analysis module comprises a rotatable holder in the form of a hollow cylinder and a plurality of strips, the plurality of strips being fixed in housings located on a circumference of the rotatable holder, said rotatable holder being removable from the urine analysis device.

37. The system of claim 27, wherein the urine analysis device comprises a pump for delivering urine from a collection port of the urine analysis device to an injector via the collection channel, the injector being configured to inject a controlled amount of urine onto a test strip.

38. The system of claim 27, wherein the urine analysis module comprises a plurality of test strips.

39. The system of claim 38, wherein the urine analysis module comprises an optical sensor.

40. The system of claim 27, wherein the urine analysis module is adapted to contact the collected urine with a detection compound, and the detection compound is a molecularly imprinted polymer.

41. The system of claim 40, wherein the molecularly imprinted polymer is incorporated into test strips in solid form.

42. The system of claim 40, wherein the molecularly imprinted polymer is functionalized with a label configured to activate when the molecularly imprinted polymer is bound to the analyte.

43. The system of 40, wherein the molecularly imprinted polymer is stored in solid form or stored in solution.

44. The system of claim 27, wherein the analysis module comprises a microfluidic chip.

45. The system of claim 27, wherein the analysis module comprises an electrochemical sensor.

46. The system of claim 27, wherein the processing unit is part of a remote device.
